(19) United States
(12) Patent Application Publication
(10) Pub. No.: US 2018/0352835 A1
(43) Pub. Date: Dec. 13, 2018

(54) COEXTRUDED LABILE COMPONENT COMPOSITIONS IN HARD CHEW FORM

(71) Applicant: Vets Plus, Inc., Knapp, WI (US)

(72) Inventors: Daniel J. DuBourdieu, Limerick, ME (US); Darwin Rajamanickam, Menomonie, WI (US); Rajiv Lall, Menomonie, WI (US)

(21) Appl. No.: 15/621,712

(22) Filed: Jun. 13, 2017

(51) Int. Cl. A23K 40/30; A23K 20/158; A23K 20/174; A23K 20/142; A23K 20/179; A61K 9/50

(52) U.S. Cl. CPC A23K 40/30; A23K 20/158; A23K 20/174; A61K 9/5057; A23K 20/179; A61K 9/5078; A23K 20/142

(57) ABSTRACT

An edible hard chew composition intended for consumption by mammals, containing labile active components such as fatty acids in an inner core surrounded by an outer core. The outer core is coated with additional stabilizers to further protect the actives from degradation in the inner core. The inner core contains additional active ingredients such as fatty acids, nutraceuticals, probiotics, prebiotics, vitamins and minerals. The invention is intended as a health supplement.

### BIBLIOGRAPHY

Complete bibliographical citations to the documents cited herein is found in the Bibliography, immediately preceding the claims.

### FIELD OF THE INVENTION

The present invention is directed to coextruded labile compounds in hard chews.

### BACKGROUND OF THE INVENTION

Numerous health issues for companion animals have underlying conditions that can be treated with antioxidant or anti-inflammatory agents. These agents are typically given in stabile drug formats using injectable or other drug delivery formats by a veterinarian or by the owner. It has been a goal to present these agents in an easier to administer format especially for longer-term maintenance use, after completing initial therapeutic dosing. This, however, has presented numerous stability challenges to the industry, due to the sensitive nature of these agents to oxygen or other degradative processes.

Labile compounds and compositions, such as polyunsaturated fatty acids (PUFAs), S-adenosylmethione, vitamins, minerals, antioxidants, amino acids, proteins, carbohydrates, coenzymes, and flavor agents, sensitive to any number of factors, can lose biological or other desired activity when unprotected in the desired treatment formats. In addition, decomposition products, degradation products, and oxidation products that result from the chemical, physical, or biological change or breakdown of labile compounds and compositions, lack the desired biological function and/or possess unwanted characteristics, such as off-flavors, undesirable odors, irritation promoting activity and the like. As used in this disclosure, the term “labile” refers to compounds, which are susceptible to alteration or destruction when exposed to other compounds or an outside force. For example, some compounds are prone to a chemical change when subjected to oxygen, heat or other forces. For companion animals, such as dogs, there is a need to introduce labile compounds and compositions, which are susceptible to chemical, physical, or biological change or breakdown, in easier to administer pharmaceutical formats.

One such format is as a hard chew treat format. Such a preparation can be presented as an adjunct therapy along with being a nutritional treat by including nutraceutical agents in the matrix. In such instances, protection of such compounds and compositions is desirable. With regard to PUFAs in particular, it is desirable to protect such lipids in food products from oxygen, trace metals and other substances, which attack the double bonds of the PUFAs. Such protection reduces the likelihood of organoleptic problems, i.e., problems relating to the senses (taste, color, odor, feel), such as off-flavors and undesirable odors, and other problems, such as loss of physiological activity. Such protection could potentially increase the shelf life of products containing them.

The methods that industry employs to protect labile agents such as PUFAs typically use microencapsulation technology. For example, US patent publication 2006/0068019 to Dalziel et al. discloses a process for coating PUFAs and creating a PUFA matrix particle. U.S. Pat. No. 7,201,923 to van Lengerich also discloses a process to create particles with a microencapsulation method of labile liquid ingredients. U.S. Pat. No. 8,221,809 to Subramanian discloses methods of encapsulated labile compound compositions using spray drying coating followed by prill coating processes. However, these microencapsulation methods are costly to use especially when desiring a variety of individual labile ingredients in the finished product. Furthermore, methods that employ coextrusion technology to create hard chews for dogs cannot justify economic costs to use multiple previously microencapsulated raw ingredients.

The present inventors have recognized the foregoing problems and that there is a need, therefore, to provide additional processes for protecting labile compounds and compositions to chemical, physical or biological change or breakdown in coextruded hard chew formats. The present invention advances the art in creating therapeutic hard chews for dogs by describing a compound and a method to utilize unprotected labile actives and stabilize them after extrusion has occurred.

### SUMMARY OF THE INVENTION

The present invention describes a cylinder-shaped hard chew matrix that has a coextruded inner core and an outer core and intended to be consumed by mammals. The inner core contains biologically active compounds such as omega-3 fatty acids and curcumin. The coextruded outer core matrix protects the contents of the inner matrix from oxidation to ensure stability of the ingredients. This advances of the art of creating hard chews with labile active ingredients by utilizing multiple labile ingredients in finished hard chews without having to microencapsulate them first. The coextrusion of labile ingredients such as fatty acids into the inner core is a novel approach to protect fatty acids from oxidation.

The entire chew is further coated with an additional stabilizer to enhance shelf life. The additional stabilizer coated on the outer surface of the invention gives additional protection from oxidation on the ends of the invention where the inner core is exposed. Using the novel inner core and final coating approach to reduce oxidation of active ingredients allows for extended shelf life of the labile compounds in the invention. The product can be used as a healthy treat supplement for mammals to support a wide range antioxidant activities and anti-inflammation processes.

There are many advantages to the method of this invention for administering fatty acids. The manufacturing process allows the inclusion of other labile compounds such as vitamins, minerals, antioxidants, amino acids, proteins, carbohydrates, coenzymes, and flavor agents, that are sensitive to any number of factors and lose biological or other desired activity when unprotected and to maintain viability and stability in the final hard chew format.

The objects and advantages of the invention will appear more fully from the following detailed description of the preferred embodiment of the invention and examples.

### DETAILED DESCRIPTION OF THE INVENTION

The present invention is primarily directed to a cylinder-shaped hard chew matrix that has a coextruded inner core and an outer core that is intended to be consumed by mammals.

### Inner Core:

The inner core contains biologically active compounds such as omega-3 fatty acids and curcumin. However, any active ingredient that provides health benefits to mammals can be incorporated into the inner core and be protected from degradation from oxygen or other processes that degrade biologically active ingredients. Other active ingredients that can be mixed into the inner core can include vitamins, minerals, herbal ingredients, nutraceuticals, drugs, and probiotics. The ingredients of the inner core will contain active ingredients and inactive binding ingredients. The inner core comprises one or more of the following ingredients.

### Flour:

The structural integrity of the present invention is supported by any one or more of the following ingredients: soy flour, wheat flour, wheat feed flour, rice flour, potato flour and other flours from cereal, grains or flours obtained upon grinding cereal grains such as corn, oats, milo, barley, and others. Other sources of ingredients include tuberous foodstuffs, such as tapioca, and the like.

### Starch:

Starch is a polymeric carbohydrate consisting of a large number of glucose units joined by glycosidic bonds. Most green plants produce this polysaccharide as an energy store. It is the most common carbohydrate in human diets and is contained in large amounts in staple foods such as potatoes, wheat, maize (corn), rice, and cassava. Starches can be converted into thermoplastic materials by heating. They are consumed by animals as a hard chew. The rheology of thermoplastic modified starch depends on the type of starch, the type of plasticizers, and the amount of plasticizers present. Starch quantities between 1% and 50%, preferably between 10% and 40%, and especially preferably between 15% and 30%, are employed in this invention. The percentages are percent by weight of the finished composition.

### Probiotics:

While there are many active ingredients found in pet supplements intended to benefit health, the use of probiotics is becoming more prevalent in use. Probiotics, which means “for life” are live microorganisms that, when eaten in regular intervals and in high enough concentrations, confer health benefits to the animal. While a delicate balance of commensal, beneficial microorganisms are already present in the gastrointestinal (GI) tract of older animals carrying out numerous and necessary functions required for the normal GI tract health of animals, supplementing with probiotics can help provide additional health benefits, especially in times of stress. In times of stress such as when diseases occur, or after surgery, or even in less traumatic situations such as kenneling, an imbalance of beneficial microorganisms can occur in the gastrointestinal tract of animals. Besides the use of antibiotics in the form of solid medications to treat these diseases, probiotics can also be provided to enhance recovery of health.

The present invention also provides probiotic compositions for animal consumption having an enhanced palatability and a beneficial effect on the gastrointestinal tract. Probiotic microorganisms in the form of live microbial nutritional supplements are recognized as conferring a beneficial effect on an animal and can be incorporated into the delivery system. Probiotic microorganisms are known to be microorganisms that beneficially affect a host by improving its intestinal microbial balance. The beneficial effects of probiotic microorganisms include activation of the immune system, prevention of the bacterial overgrowth by pathogens, prevention of diarrhea and/or restoration of intestinal flora.

Suitable probiotics for including in the viscoelastic mass include but are not limited to microorganisms of the genera Aspergillus, Trichoderma, Bacillus, Bacteriodies, Bifidobacterium, Lactobacillus, Leuconostoc, Streptococcus, Pediococcus, Propionibacterium, Saccharomyces, Enterococcus, and Escherichia. Suitable species of these genera include but are not limited to Aspergillus oryzae, Aspergillus niger, Trichoderma longbranchiatum, Bacillus subtilus, Bacteriodies thetaiotaomicron, Bacteriodies longum, Bifidobacterium longus, Bifidobacterium infantis, Lactobacillus acidophilus, Lactobacillus acidophilus, Lactobacillus bulgaricus, Lactobacillus casei, Lactobacillus lactis, Lactobacillus plantarum, Lactobacillus rhamnosus, Lactobacillus sakei, Leuconostoc mesenteroides, Leuconostoc cremoris, Streptococcus diacetylactis, Streptococcus florentinus, Streptococcus thermophilus, Pediococcus acidilactici, Pediococcus pentosaceus, Propionibacterium freudenreichii, Saccharomyces boulardii, Enterococcus faecalis, Enterococcus faecium, Escherichia coli Nissle 1917. The probiotic may be included in a spray dried or freeze-dried form in an amount of from about 0 to 1×10¹¹ CFU/g of the composition.

### Prebiotics:

While probiotics are helping the animal, there is a series of compounds that are helpful to the probiotics themselves called prebiotics (before life). Prebiotics are indigestible ingredients typically made out of oligosaccharide dietary fibers such as Fructo-oligosaccharides (FOS), inulin, Manna-oligosaccharides (MOS), beet pulp, psyllium, cellulose and gum arabic. These compounds selectively stimulate the growth and activity of the probiotic bacteria, which colonize the colon. Probiotic bacteria, such as Lactobacillus and Bifidobacterium can utilize prebiotic fiber sources as a source of nutrition. However, enteric pathogenic bacteria, such as Salmonella and E. coli are unable to utilize these fiber sources.

Prebiotics can be delivered alone or in combination with probiotic bacteria in the delivery systems. Prebiotics comprise carbohydrates, generally oligosaccharides, and have the ability to resist hydrolysis by enzymes in the animal digestive tract and thus can reach the colon undegraded to provide a carbohydrate substance particularly suited to growth of probiotic bacteria. Suitable examples of prebiotics include inulin, lactulose, lactitol, fructooligosaccharides, galactooligsaccharides, xylooligosaccharides, isomaltooligosaccharides, mannaoligosaccharides, lactosucrose, cereal fibers, soy oligosaccharides, raffinose, beet pulp, psyllium, cellulose, and gum arabic. Prebiotics are included in the composition typically in an amount between 0% and 10% w/w, preferably 0.5% and 6% w/w, and most preferably 1% and 3% w/w.

### Minerals:

More than 18 mineral elements such as sodium, calcium, zinc, manganese, copper, molybdenum and others are believed to be essential for mammals. Macrominerals are required by the animal in the diet in larger amounts and microminerals or trace elements in much smaller amounts. Minerals can be provided in therapeutically effective amounts to inner core composition. Water and oxygen can interact with minerals and form oxides and thus change the original nature. Preferred microminerals in the invention can include iron from 5 to 30 mg/Kg of final product, copper from 1 to 7.3 mg/Kg of final product, manganese from 1 to 5.0 mg/Kg of final product, zinc from 10 to 80 mg/Kg of final product, iodine from 0.1 to 0.88 mg/kg of final product, and selenium from 0.05 to 0.35 mg/Kg of final product

### Proteins and Amino Acids

Proteins are large biomolecules, or macromolecules, consisting of one or more long chains of amino acid residues. Proteins perform a vast array of functions within organisms, including catalyzing metabolic reactions, DNA replication, responding to stimuli, and transporting molecules from one location to another. Proteins can be degraded in the presence of extremes of pH or heat. Proteins such as transferrin and lactic dehydrogenase can be degraded oxygen. Certain amino acids, such as glutamine, are also prone to degradation in the presence of water.

### Polyunsaturated Fatty Acids (PUFAs)

Due to commonality for their mode of action in health issues, PUFAs and omega-3 fatty acids are used in managing many veterinary diseases including neoplasia, dermatologic disease, hyperlipidemia, cardiovascular disease, renal disease, gastrointestinal disease and orthopedic disease. Omega-3 and omega-6 fatty acids (also called ω-3 and ω-6 fatty acids or n-3 and n-6 fatty acids) are polyunsaturated fatty acids (PUFAs) with a double bond (C═C) at the third or sixth carbon atom from the end of the carbon chain for the omega 3 and omega 6, respectively. The fatty acids have two ends, the carboxylic acid (—COOH) end, which is considered the beginning of the chain, thus “alpha”, and the methyl (CH3) end, which is considered the “tail” of the chain, thus “omega.” The way in which a fatty acid is named is determined by the location of the first double bond, counted from the methyl end, that is, the omega (ω-) or the n-end.

The three types of omega-3 fatty acids involved in mammalian physiology are α-linolenic acid (ALA), found in plant oils, eicosapentaenoic acid (EPA), and docosahexaenoic acid (DHA), both commonly found in marine oils. The main source of these fatty acids has been from fish, krill oil, or plant source. Regardless of the source of the PUFAs, they are prone to oxidation and therefore degradation. Finding ways to protect the PUFAs has been a major challenge. However, krill-sourced omega-3 fatty acids are proving to have unique mechanisms of action and efficacy in animals for numerous health benefits and better stability due to the presence of antioxidants, such as astaxanthin, in hill. Other sources of omega-3 fatty acids include marine algae and phytoplankton. Common sources of plant oils containing the omega 3 ALA fatty acid include walnut, edible seeds, clary sage seed oil, algal oil, flaxseed oil, Sacha Inchi oil, Echium oil, and hemp oil, while sources of animal omega-3 EPA and DHA fatty acids include fish oils, egg oil, squid oils, and hill oil. Omega 3 and omega 6 fatty acids are selected from the group consisting of plant oils, fish oils, animal oils, algea sources and crustacean sources. Evening primrose oil is an excellent source of omega 6 polyunsaturated fatty acids. Linoleic acid (18:2, n-6), the shortest-chained omega-6 fatty acid, is one of many essential fatty acids and is categorized as an essential fatty acid because mammals cannot synthesize it. Mammalian cells lack the enzyme omega-3 desaturase and therefore cannot convert omega-6 fatty acids to omega-3 fatty acids.

The amount of omega 3 and omega 6 polyunsaturated fatty acids included in the viscoelastic mass can be adapted to the specific needs of the target animal. As an example, omega 3 and omega 6 polyunsaturated fatty acids may be included in an amount of from about 0.001% to about 25% w/w of the viscoelastic mass, preferably from about 1.0% to about 20.0% w/w, and more preferably from about 8.0% to about 15.0% w/w.

### Antioxidants

Examples of suitable antioxidants include astaxanthin, alpha-tochopherol, alpha-tochopherol acetate, butylated hydroxytoluene (BHT), ascorbic acid, tocopherol and propyl gallate and mixtures thereof. The antioxidant is present in amounts ranging from about 0% to about 0.3%, preferably from about 0.025% to about 0.2%, and most preferably from about 0.05% to 0.15% percent by weight.

Astaxanthin is a very potent antioxidant with anti-inflammatory properties. The safety, bioavailability and effects of astaxanthin on oxidative stress and inflammation are well known on the pathophysiology of atherosclerotic cardiovascular events. There is evidence of a reduction in biomarkers of oxidative stress and inflammation with astaxanthin administration (Fasset). Animal models that have been used in studies with krill include obesity, depression, myocardial infarction, chronic low-grade and ulcerative inflammation (Burri). There is an increased tissue uptake of the long-chain omega-3 PUFAs eicosapentaenoic acid (EPA) and docosahexaenoic acid (DHA) in krill, since hill omega-3 are found in highly bioavailable phospholipid forms, as compared to the less bioavailable fish triglyceride form (Ulven). Krill is known to safely benefit health concerns in animals affecting the joints, skin, liver, kidney, muscle and brain and help alleviate metabolic dysfunction and its associated cardiovascular disease risk.

### Anti-Inflammatory Agents

A novel approach of the current invention as an adjunct therapy along with being a nutritional treat is to combine PUFAs with an anti-inflammatory agent. Inflammation is associated with many disease and physical conditions. The anti-inflammatory agents can be of an herbal/phytobotanical nature or they can be considered as classical drugs.

Certain herbs have been found to be highly beneficial to fight inflammatory conditions as they contain specific compounds that are biologically active. For example, turmeric (Curcumin longa) that contains curcumin, garlic (Allium sativum), cinnamon (Cinnamonum spp.), ginger, (Zingeber officianale), Roman chamomile, Echinacea, red clover, goldenseal, Vitex (Chaste tree), black pepper, clove are among the phytobotanicals that have known anti-inflammatory activity that can be effective in the invention.

Curcumin is the active ingredient of turmeric. It is widely used as a safe kitchen spice and food colorant throughout the world. It is a complex molecule with multiple biological targets and different cellular effects. Its molecular mechanisms of action have been extensively investigated (Epstein) and are known to have anti-inflammatory, antioxidant and anti-cancer properties.

Drugs such as aspirin, Diclofenic, Ketorolac, Asteminofen, Flunixin, Megalum, Meloxicam, Nabumetane, Nimesulid, Carpofren. Celecoxib, and Rofecoxib are known to specifically inhibit either cyclooxygenases 1 or 2 or both and thus effectively reduce inflammation. The current invention can select from what are considered anti-inflammatory drugs in addition to anti-inflammatory phytobotanicals.

A preferred anti-inflammatory agent is turmeric-containing curcumin. The anti-inflammatory agent may be included in an amount of from about 0.01% to about 10% w/w of the composition, such as from about 0.1% to about 5% w/w or from about 0.5% to about 1.5% w/w.

### Vitamins

Vitamin deficiencies can occur if poor quality food is provided to animals. Likewise if the animal is under stress, vitamin deficiencies can also occur. Ill or recovering pets that may have a poor appetite typically need a vitamin supplement since they are not receiving their daily requirements through the food they eat. Other situations for dogs, like stress from travel, showing, training, hunting, breeding or lactation, can also utilize vitamin supplementation. Vitamins are necessary for literally tens of thousands of different chemical reactions in the body. They often work in conjunction with minerals and enzymes to assure normal digestion, reproduction, muscle and bone growth and function, healthy skin and hair, clotting of blood, and the use of fats, proteins, and carbohydrates by the body.

For example, vitamin E isomers (mixed tocopherols) are antioxidants that helps protect animals from free radical damage. However, older animals tend to absorb fewer vitamins, minerals, and electrolytes through the intestinal tract, and lose more of them through the kidneys and urinary tract. In addition, some older animals eat less, due to conditions such as oral disease, and may not receive their daily needs of vitamins and minerals. These same older animals are the ones that will also be given solid medications to help overcome vitamin deficiencies.

Vitamins may be provided according to the nutritional requirements of the target animal, and may be provided as an element of other oils utilized in the present invention, such as, for example, canola oil, corn oil, soybean oil and vegetable oil. In the present invention, the vitamins can be hydrosoluble or liposoluble vitamins. Examples of such vitamins include, but are not limited to, vitamin A, vitamin D, vitamin E, vitamin K, vitamin B and vitamin C.

The dosage of the vitamins in the delivery system can be adapted to specific needs. Typical dosages include between about 0% and 10% w/w, preferably 0.01% and 5% w/w, and most preferably 0.5% and 1% w/w.

### S-Adenosylmethionine

S-Adenosyl methionine (SAME) is a common co-substrate involved in methyl group transfers, trans-sulfuration and amino-propylation. Some research, including multiple clinical trials, has indicated taking SAME on a regular basis may help fight depression, liver disease, and the pain of osteoarthritis. SAME has been studied in the treatment of osteoarthritis, wherein the substance reduces the pain associated with the disease. Although an optimal dose has yet to be determined, SAME appears as effective as the non-steroidal anti-inflammatory drugs. A proposed dosage of the S-Adenosylmethionine will be given at 10 mgs per pound of body weight of a dog. This is between about 0% and 10% w/w, preferably 0.01% and 5% w/w, and most preferably 0.5% and 2.5% w/w of the invention.

### Emulsifiers

Emulsifiers which can be employed are selected from groups including nonionic surfactants, e.g., polyoxyethylated castor oil, polyoxyethylated sorbitan monooleate, sorbitan monostearate, ethyl alcohol, glycerol monostearate, polyoxyethyl stearate, alkylphenol polylglycol ethers; and ampholytic surfactants, e.g., disodium N-lauryl-B-iminodipropionate or lecithin; or anionic surfactants, such as sodium lauryl sulphate, fatty alcohol ether sulphates, mono-dialkyl polyglycol ether orthophosphoric ester monoethanolamine salt. The quantities employed here preferably amount to anywhere between 0 and 20% by weight based on the total amount of constituents. Quantities of from 4% to 16% by weight are preferred, and quantities of from 6% to 8% by weight are especially preferred.

A preferred emulsifier is lecithin, such as soy lecithin. The emulsifier may be included in an amount of from about 0% to about 20% w/w of the composition, preferably from about 4% to about 16% w/w, and more preferably from about 4.5% to about 5.5% w/w.

### Plasticizer

In order to provide an edible chew, plasticizing agents are preferred for the composition of the present invention. Examples include glycerol and propylene glycol, and wetting agents such as cetyl alcohol and glycerol monostearate. Glycerol is a preferred plasticizer useful in maintaining the softness of the edible chew over the shelf life of the product.

Amounts of between 0 and 50% w/w can be used in the present invention, with preferred amounts between 2% and 25% w/w and especially preferred amounts between 5% and 18% w/w.

### Flavorings and Sweeteners

Flavorings and sweeteners are preferably present in the composition of the present invention. All flavorings and sweeteners must be of at least food grade quality. The composition can include such additives as sweeteners and flavorings. Sweeteners can be selected from a wide variety of suitable materials known to the art.

Representative and non-limiting examples of sweeteners include xylose, ribose, sucrose, mannose, galactose, fructose, dextrose, maltose, and mixtures thereof.

Natural sweeteners such as sugar and molasses may be used. In addition to natural flavorings such as chicken flavor, other non-animal flavorings can include, for example, anise oil, carob, peanuts, fruit flavors, other sweeteners such as honey and maple syrup, herbs such as parsley, celery leaves, peppermint, spearmint, garlic, or combinations thereof.

Natural and synthetic flavor oils can also be used. Examples include spearmint oil, peppermint oil, cinnamon oil, wintergreen oil, citrus oils, including lemon, orange, grape, lime and grapefruit, and other fruit essences including apple, strawberry, cherry, pineapple, and others that are familiar to the art.

Quantities of between 0% and 20% w/w, preferably between 0.1% and 10% w/w, and especially preferably between 0.5% and 1% w/w are employed in this invention. The percentages are percent by weight of the finished composition.

### Nutraceuticals

A nutraceutical can be defined as, “a food (or part of a food) that provides medical or health benefits, including the prevention and/or treatment of a disease.” Suitable nutraceuticals are derived from botanical or animal sources or microbial sources. Examples of nutraceuticals from botanical sources include phytochemicals such as flavonoids, isoprenoids, proteins, bioflavonoids, carotenoids and others. It should be noted that some of these nutraceuticals from botanical sources can be bitter tasting and therefore not very palatable. Examples of nutraceuticals from animal sources include glucosamine and chondroitin sulfate. The amounts in the composition can range from 0.001% w/w to 50% w/w of the composition.

### Preservatives

A preservative such as potassium sorbate, methylparaben, propylparaben, sodium benzoate or calcium propionate may be included in order to retard growth of microorganisms and fungi. Preservatives are added in an amount between 0% and 1% by weight, preferably between about 0.05% and 0.5% by weight and most preferably between about 0.9% and 0.11% by weight.

### Amounts of Components

The amounts of each of the components in the final product may be varied depending upon the nature of the freeze-dried protein, the weight and condition of the animal to be treated, and the unit dosage desired. Those of ordinary skill in the art will be able to adjust dosage amounts as required.

### Color of Inner Core:

The color of the inner core can be of any color and will depend on the particular ingredients included in the inner core mix.

### Outer Core:

The coextruded outer core matrix protects the active ingredients of the inner matrix from oxidation to ensure stability of the ingredients. The outer core inactive ingredients work as binding agents to give mass and bulk to the invention. The outer core matrix includes one or more of the following ingredients.

### Base Powder:

The outer core matrix preferably comprises a base powder. The base powder generally provides structural integrity to the matrix. The base powder may comprise a plant powder, an animal powder, or both a plant and an animal powder. Plant powders are powders derived from plants, such as flours or other powders. The flours may be whole flours or flours that have had fractions, such as the germ fraction or the husk fraction, removed. Non-limiting examples of suitable plant powders include soy flour, wheat flour, whole wheat flour, whole wheat fine flour, wheat feed flour, wheat gluten, pre-gel wheat flour, potato flour, corn flour, oat flour, soy protein concentrate, oat flour or powder, barley powder or flour, brown rice flour or powder, dried whey powder, carrot powder, cherry powder, pineapple powder, and alfalfa herb powder. Animal powders are powders derived from animals and can include dehydrated meat byproducts, such as liver powder. In a preferred version of the invention, the base powder comprises an animal powder and a plant flour, which can be mixed with a fluid lubricant. The powder is preferably included in an amount of from about 10% to about 70% w/w of the matrix.

### Starch:

The outer core matrix mass may include a starch. For the outer core matrix, “starch” refers to any substance comprised of more than about 80%, 90%, 95%, or even 99% amylase and amylopectin by weight. Starches from various sources are known in the art. Suitable starches can be obtained from tuberous foodstuffs, such as potatoes, tapioca, and the like. Other suitable starches can be obtained upon grinding cereal grains such as corn, oats, wheat, milo, barley, rice, and others. The starch may be included in an amount of from about 0.1% to about 25% w/w of the viscoelastic mass, such as from about 1% to about 15% w/w or from about 5% to about 9% w/w.

### Softening Agent:

In certain versions, the outer core matrix comprises a softening agent, wherein the softening agent comprises glycerol in an amount of from about 9% to about 14% w/w of the matrix.

Other ingredients, such as carboxymethyl cellulose, xanthan gum, brewer's yeast and flaxseed, can be included in the outer core matrix.

### Glycerol:

Glycerol can be used as a softening agent in the both the inner and outer core. Glycerol prevents the matrix from being too hard as it can prevent the matrix from drying out if water evaporates. Propylene glycol can be used as an alternative softening agent.

### Carboxymethyl Cellulose:

Carboxymethyl cellulose is used as a thickener and prevent syneresis of the matrix. It helps to increase the viscosity of the matrix and help stabilize the invention. There are many varieties of cellulose that can used in the invention including methyl cellulose.

### Xanthan Gum:

Xanthan gum is a polysaccharide and a thickening agent. It is used as a stabilizer to prevent ingredients from separating in the matrix of the invention. There a numerous gum alternatives to xanthan gum that can work in the invention including guar gum, ghatti gum, guaiac gum, and tragacanth gum.

### Brewer's Yeast:

Brewer's yeast plays roles both as an inactive and as an active ingredient. As an inactive in the base formulation, it is a rich source of protein that brings stability from structural and rheological transformation of biopolymers during extrusion. This allows keeping the final product more rigid. AS an active ingredient, Brewer's yeast is also a good source of vitamin B-complex, chromium, selenium, omega fatty acids, and antioxidants. The active nature of Brewer's yeast can help improves the immune system and improve the skin. There are numerous varieties of yeast that can also work in the invention including Baker's yeast and spent yeast.

### Flaxseed:

Flaxseed is an active ingredient source of omega-3 fatty acid that has been shown to have a beneficial effect on inflammatory disorders of the skin and coat. In addition to the omega fatty acids, flax seed contains alpha-linoleic acid, which offers benefits to animal's immune system, arthritis or other joint problems.

A preferred example of these binding agents includes rice flour (34.17% w/w), potato starch (25.41% w/w), glycerin (16.85% w/w), carboxymethyl cellulose (2.78% w/w), oat flour (1.79% w/w), xanthan gum (1.37% w/w), brewer's yeast (0.88% w/w), and flaxseed (0.88% w/w) that are mixed together. Preservatives L+lactic acid (0.5% w/w), methylparaben (0.1% w/w), propylparaben (0.1% w/w), sorbic acid (0.1% w/w), sodium bicarbonate (0.05% w/w) and chicken flavor powder (0.86% w/w) are then mixed in. Water (14.17% w/w) is then mixed in to create the formulation of the outer core.

### Stabilizers:

The invention utilizes stabilizing compounds that do not interact with oxygen. These stabilizing compounds can be sprayed onto the extruded pieces of the invention after they exit the extruder. The stabilizing agents can also be coated onto the extruded pieces by placing the extruded pieces into a bath of stabilizing agent. Examples of stabilizing agents that are effective include hydrogenated fats.

Fats, from animal or plant sources, are esters of three fatty acid chains and the alcohol glycerol. They are either in a liquid format or in a solid format. The term oil normally refers to a fat type with short or unsaturated fatty acid chains that is a liquid at room temperature, while generally the term fat refers to fat types that are solids at room temperature. The fatty acids in any fat type are made of chains of carbon that have varying amounts of hydrogen attached to the carbon. If the carbon chain is an alkene and has a double bond, it is described as being unsaturated because there is less hydrogen present. If the carbon chain is an alkane then the chain is considered to be saturated in hydrogen, and therefore is known as a hydrogenated fat. An unsaturated fat can be made in to a saturated fat via hydrogenation reactions.

Vegetable oils are commonly referred to as “polyunsaturated.” This simply means that there are several double bonds present. Vegetable oils may be converted from liquids to solids by hydrogenation reactions. Margarines and shortenings are hydrogenated in this way to make them solid or semi-solids. Vegetable oils, which have been partially hydrogenated, are partially saturated so the melting point increases to the point where a solid is present at room temperature. The degree of hydrogenation of unsaturated oils controls the final consistency of the hydrogenated fat. Lard from animal sources is a type of fat that is solid already. The present invention can utilize partially or fully hydrogenated fats that are from any animal source or plant source that are solids or semi solid at room temperature. Non-limiting examples of hydrogenated fats, which may be used in this invention, include vegetable oil, margarines, shortenings and animal lard.

Another stabilizing agent that is effective in the invention is gelatin. Gelatin is a translucent, colorless, brittle (when dry), flavorless food derived from collagen obtained from various animal raw materials. Gelatin is an irreversibly hydrolyzed form of collagen, wherein the hydrolysis results in the reduction of protein fibrils into smaller peptides, which will have broad molecular weight ranges associated with physical and chemical methods of denaturation, based on the process of hydrolysis.

Another stabilizing agent that is effective in the invention is wax. Waxes are a diverse class of organic compounds that are hydrophobic, malleable solids near ambient temperatures. They include higher alkanes and lipids, typically with melting points above about 40° C. (104° F.), melting to give low viscosity liquids. Waxes are insoluble in water but soluble in organic, nonpolar solvents. Natural waxes of different types are produced by plants and animals and occur in petroleum. Waxes are organic compounds that characteristically consist of long alkyl chains. Synthetic waxes are long-chain hydrocarbons (alkanes or paraffins) that lack substituted functional groups. Natural waxes may contain unsubstituted hydrocarbons, such as higher alkanes, but may also include various types of substituted long chain compounds, such as fatty acids, primary and secondary long chain alcohols, ketones and aldehydes. They may also contain esters of fatty acids and long chain alcohols.

Other stabilizers that are effective in the invention include glycerol and propylene glycol.

### Preparation:

All weighed dry ingredients are placed in a mixer and mixed for 5 minutes or until homogenized. Liquid ingredients are added on top of the dry mix and mixed for approximately five more minutes or until homogenized.

In a similar manner for the outer core formulation, ingredients are mixed together. The mixtures for the inner core and outer core are loaded into a standard hot melt coextrusion machine. These coextrusion machines are well known in the field. Temperatures are set for the varying parts of the machine and the invention is extruded. Once extruded, the cylinder is cut to length, coated with final stabilizer, and packaged.

Once the inner and outer core is coextruded and cut to desired length, the outer surface of the invention subjected to further stabilization by coating the surface of the invention with stabilizers. The stabilizers can be sprayed onto the surface of the cylinder or the cylinder can be dipped into a bath of stabilizer.

The function of the stabilizer is to coat the ends of the cylinder where the inner core is exposed to air and protect the ends of the inner core active ingredients from oxidation. Stabilizers that can be used to protect from oxidation include hydrogenated fat, wax, gelatin, glycerol, propylene glycol and other such suitable materials that block oxygen or other degradative processes from the exposed inner core surface.

### Density:

The density of the invention is dependent on the specific types and amounts of inactive binding agents used in the formulation. Typically, the density will be hard enough so that it takes a bit of hand pressure from a normal human fingernail to leave an indentation.

### Dimension of Product:

The outer dimensions of the cylinder can be of any size achievable with standard hot melt extrusion equipment. Typical diameter of the cylinder of the invention is one inch. The length of the cylinder can be any length desired; however, the typical length is about 4 inches long. Length and diameter are dictated by what a typical dog might consume.

### Appearance:

Depending on the die used to coextrude the outer core, the outer shape can be square, circular, star, or any other geometric shape desired. The color of the outer core will depend on the particular ingredients used in the outer core mixture.

The chew can be used by any animal, including mammals, in need of nutritional supplementation and/or a particular medication. Non-limiting examples of suitable animals include humans, dogs, cats, horses, cows, pigs, goats, and sheep, among others.

### EXAMPLES

The following examples are designed to illustrate the invention without limiting the same.

### Example 1

Ingredients used as inactive binding agents are mixed together for the inner core matrix. Potato flour (43.07% w/w), potato starch (11.32% w/w), glycerin (7% w/w), and lecithin (5% w/w) are mixed together in a container. Active ingredients of fish oil (20% w/w), vitamin E (0.15% w/w), and biotin (0.03% w/w) are then mixed in.

Preservatives of L+lactic acid (0.5% w/w), methylparaben (0.1% w/w), propylparaben (0.1% w/w), sorbic acid (0.1% w/w), and sodium bicarbonate (0.05% w/w) are then mixed in. Natural red color (0.5% w/w) and water (12.18% w/w) are subsequently mixed in to create formulation of inner core.

Ingredients used as inactive binding agents are mixed together for the outer core matrix. Rice flour (34.17% w/w), potato starch (25.41% w/w), glycerin (16.85% w/w), carboxymethyl cellulose (2.78% w/w), oat flour (1.79% w/w), xanthan gum (1.37% w/w), Brewer's yeast (0.88% w/w), and Flaxseed (0.88% w/w) are mixed together. Preservatives L+Lactic acid (0.5% w/w), Methylparaben (0.1% w/w), Propylparaben (0.1% w/w), sorbic acid (0.1% w/w), sodium bicarbonate (0.05% w/w) and chicken flavor powder (0.86% w/w) are then mixed in. Water (14.17% w/w) is then mixed in to create the formulation of the outer core.

The inner and outer core product is loaded into the extrusion machine and parameters for extrusion are set for the outer core matrix.

### Extruder Barrel Temperatures for Outer Core Matrix:

Feed Zone: 60° C.
Transition Zone 1: 90° C.
Transition Zone 2: 80° C.
Extrusion Zone: 60° C.
Feed screw speed: 5 Hz
Barrel screw speed: 16 Hz
   Extrusion parameters for the inner core are set matrix also set as follows:

### Extruder Barrel Temperatures for Inner Core Matrix:

Feed Zone: 60° C.
Transition Zone 1: 100° C.
Transition Zone 2: 90° C.
Extrusion Zone: 70° C.
Feed screw speed: 1 Hz
Barrel screw speed: 28 Hz

The inner and outer cores are coextruded and the resulting cylinder is cut to 4-inch length. The 4-inch pieces are coated with gelatin to cover the exposed end of the inner core and further stabilize the labile ingredients in the inner core. The pieces are air-dried and then packaged in screw-capped plastic jars.

### Example 2

Ingredients used as inactive binding agents are mixed together for the inner core matrix. Rice flour (23.38% w/w), potato starch (17.59% w/w), glycerin (16.79% w/w), brewer's yeast (1% w/w) and xanthan gum (1% w/w) were mixed together in a container. Active ingredients of krill meal (4% w/w), salmon meal (16% w/w), and flaxseed (1% w/w) were added to the container. Preservatives of L+lactic acid (1% w/w), methylparaben (0.1% w/w), propylparaben (0.1% w/w) and sorbic acid (0.5% w/w) were added. Sodium bicarbonate (0.04% w/w), flavor-bacon flavor powder (1% w/w) and natural red color (1.06% w/w) were then added. Water (15.42% w/w) was added and the entire matrix was mixed.

The inner and outer core product is loaded into the extrusion machine and parameters for extrusion are set for the outer core matrix.

### Extruder Barrel Temperatures for Outer Core Matrix:

Feed Zone: 60° C.
Transition Zone 1: 90° C.
Transition Zone 2: 80° C.
Extrusion Zone: 60° C.
Feed screw speed: 5 Hz
Barrel screw speed: 16 Hz
   Extrusion parameters for the inner core are set matrix also set.

### Extruder Barrel Temperatures for Outer Core Matrix

Feed Zone: 60° C.
Transition Zone 1: 100° C.
Transition Zone 2: 90° C.
Extrusion Zone: 70° C.
Feed screw speed: 1 Hz
Barrel screw speed—28 Hz

The inner and outer cores are coextruded and resulting cylinder is cut to 4-inch length. The 4-inch pieces are sprayed with melted hydrogenated fat to cover the exposed end of the inner core. The pieces are air-dried and then packaged in screw capped plastic jars.

### Example 3

An extruded sample of only the unprotected inner core matrix of Example 1 was analyzed by gas chromatography for the EPA and DHA fatty acid content at time day 14 after being held at 25° C., 37° C. and 50° C. Values are noted in Table 1. Samples of the completed protected invention stored at temperatures of 25° C., 37° C. and 50° C. for 14 days for accelerated stability. These samples were also analyzed for EPA and DHA content as before. It was found that degradation occurred in the unprotected inner core EPA and DHA fatty acid content relative to the protected complete invention. This indicates that the outer core and final coating with stabilizer helps to protect EPA and DHA found in the inner core from degradation.

TABLE 1

| | EPA | DHA |
|---|---|---|
| | (mg/g) | (mg/g) |
| Unprotected for 14 days | | |
| 25° C. | 2.3 | 1.8 |
| 37° C. | 1.1 | 1.0 |
| 55° C. | 0.7 | 0.4 |
| Protected for 14 days | | |
| 25° C. | 2.7 | 2.5 |
| 37° C. | 1.4 | 1.1 |
| 55° C. | 1.3 | 1.0 |

### Example 4

Ingredients used as inactive binding agents are mixed together for the inner core matrix. Rice flour (23.38% w/w), potato starch (17.59% w/w), glycerin (16.79% w/w), brewer's yeast (1% w/w) and xanthan gum (1% w/w) were mixed together in a container.

Active ingredients of hill meal (4% w/w), salmon meal (15% w/w), turmeric (1% w/w) and flaxseed (1% w/w) were added to the container. Preservatives of L+lactic acid (1% w/w), methylparaben (0.1% w/w), propylparaben (0.1% w/w) and sorbic acid (0.5% w/w) were added. Sodium bicarbonate (0.04% w/w), bacon flavor powder (1% w/w), natural red color (1.06% w/w) were then added. Water (15.42% w/w) was added and the entire matrix was mixed.

The inner and outer core product is loaded into the extrusion machine and parameters for extrusion are set for the outer core matrix.

### Extruder Barrel Temperatures for Outer Core Matrix

Feed Zone: 60° C.
Transition Zone 1: 90° C.
Transition Zone 2: 80° C.
Extrusion Zone: 60° C.
Feed screw speed: 5 Hz
Barrel screw speed: 16 Hz
   Extrusion parameters for the inner core matrix were also set as follows.

### Extruder Barrel Temperatures for Outer Core Matrix

Feed Zone: 60° C.
Transition Zone 1: 100° C.
Transition Zone 2: 90° C.
Extrusion Zone: 70° C.
Feed screw speed: 1 Hz
Barrel screw speed: 28 Hz

The inner and outer cores are coextruded and the resulting cylinder is cut to a 4-inch length. The 4-inch pieces are sprayed with melted hydrogenated fat to cover the exposed end of the inner core. The pieces are then air-dried and then packaged in screw capped plastic jars.

### Example 5

Ingredients used as inactive binding agents were mixed together for the inner core matrix. Brown rice flour (23.38%), oat flour (12.59%), corn syrup (14.79%), liquid lecithin (2.79%) and brewer's yeast (1%) were mixed together in a container.

Active ingredients for the inner core of hill meal (4% w/w), salmon meal (15% w/w), turmeric containing curcumin (1% w/w) were added to the container. Preservatives of L+lactic acid (1% w/w), methylparaben (0.1% w/w), propylparaben (0.1% w/w), and Sorbic acid (0.5% w/w) were added. Sodium bicarbonate (0.04% w/w) Flavor-Bacon flavor powder (1% w/w), Natural red color (1.06% w/w) were then added. Water (15.42% w/w) was added and the entire matrix was mixed for the composition of the inner core.

Cornstarch (33.78%), potato flour (8.09%), liquid sorbitol (16.85%), carboxymethyl cellulose (2.79%), and xanthan gum (1.37%), water (37.12) were mixed together for form binders for the outer core.

The inner and outer core product is loaded into the extrusion machine and parameters for extrusion are set for the outer core matrix.

Extruder barrel temperatures for outer core matrix:
Feed Zone: 60° C.
Transition Zone 1: 90° C.
Transition Zone 2: 80° C.
Extrusion Zone: 60° C.
Feed screw speed: 5 Hz
Barrel screw speed: 16 Hz

### Extrusion Parameters for the Inner Core are Set Matrix Also Set at the Following Parameters:

Feed Zone: 60° C.
Transition Zone 1: 100° C.
Transition Zone 2: 90° C.
Extrusion Zone: 70° C.
Feed screw speed: 1 Hz
Barrel screw speed: 28 Hz

The inner and outer cores are coextruded and resulting cylinder is cut to 4-inch length. The 4-inch pieces are stabilized by coating with a 5% gelatin solution to cover the exposed end of the inner core. The pieces are then air-dried and then packaged in screw capped plastic jars.

There are several variations, which can be practiced in the scope of this invention. The invention may be provided in a variety of shapes and sizes of the freeze-dried meat. The invention may include a variety of other additives such as probiotics, prebiotics, vitamins and minerals.

Any version of any component or method step of the invention may be used with any other component or method step of the invention. The elements described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference in their entirety to the same extent as if each individual reference were specifically and individually indicated as being incorporated by reference. In case of conflict between the present disclosure and the incorporated references, the present disclosure controls.

The devices, methods, compounds and compositions of the present invention can comprise, consist of, or consist essentially of the essential elements and limitations described herein, as well as any additional or optional steps, ingredients, components, or limitations described herein or otherwise useful in the art.

While this invention may be embodied in many forms, what is described in detail herein is a specific preferred embodiment of the invention. The present disclosure is an exemplification of the principles of the invention is not intended to limit the invention to the particular embodiments illustrated. It is to be understood that this invention is not limited to the particular examples, process steps, and materials disclosed herein as such process steps and materials may vary somewhat. It is also understood that the terminology used herein is used for the purpose of describing particular embodiments only and is not intended to be limiting since the scope of the present invention will be limited to only the appended claims and equivalents thereof.

### BIBLIOGRAPHY

Burri L. and Line Johnsen. Krill Products: An Overview of Animal Studies for efficacy. Nutrients 2015, 7, 3300-3321.
Epstein, J.; et al., “Curcumin as a therapeutic agent: the evidence from in vitro, animal and human studies,” British Journal of Nutrition, 2010, 103, 11, pp 1545-1557.
Fassett R. G. and Jeff S. Coombes, “Astaxanthin: A Potential Therapeutic Agent in Cardiovascular Disease,” Marine Drugs, 2011, 9(3): 447-465.
Ulven S M and K B Holven, “Comparison of bioavailability of krill oil versus fish oil and health effect,” Vascular Health and Risk Management, 2015:11 511-524.
US patent publication 2006/0068019 to Dalziel et al.
U.S. Pat. No. 7,201,923 to van Lengerich
U.S. Pat. No. 8,221,809 to Subramanian

What is claimed:

1. A hard chew matrix composition for consumption by mammals, comprising a coextruded inner core matrix and an outer core matrix, wherein the inner core matrix comprises at least one biologically active compound supported in a flour base and the coextruded outer core matrix comprises materials to protect the inner core matrix.

2. The hard chew matrix of claim 1 wherein the biologically active compounds are selected from at least one of the following: vitamins, at least one antioxidant, minerals, herbal ingredients, nutraceuticals, drugs, probiotics, prebiotics, S-adenosylmethione polyunsaturated fatty acids and curcumin.

3. The hard chew matrix of claim 2 wherein the probiotics are present in spray dried or freeze-dried form in an amount from about 0 to 1×10¹¹ CFU/g of the composition.

4. The hard chew matrix of claim 2 wherein the probiotics are selected from Bacillus species.

5. The hard chew matrix of claim 1 further comprising a stabilizer coating surrounding the surface of the outer core matrix to enhance shelf life.

6. The hard chew matrix of claim 5 wherein the stabilizer is selected from the group consisting of hydrogenated fat, vegetable oil, margarines, shortenings, animal lard, gelatin, wax, glycerol and propylene glycol.

7. The hard chew matrix of claim 6 wherein the hydrogenated fat is selected from the group consisting of vegetable oil, margarines, shortenings and animal lard.

8. The hard chew matrix of claim 1 wherein the flour base is selected from the materials consisting of soy flour, wheat flour, wheat feed flour, rice flour, potato flour and cereal flour.

9. The hard chew matrix of claim 2 wherein the prebiotics are selected from the group consisting of Fructo-oligosaccharides, inulin, Lactulose, Lactitol, Galacto oligosaccharides Xylooligosaccharides Isomaltooligosaccharides Lactosucrose, Cereals fibers, Soy oligosaccharides Raffinose Fructo-oligosaccharides in amounts ranging from 0 to 10% w/w of the composition.

10. The hard chew matrix of claim 2 wherein the minerals comprise iron in an amount from 5 to 30 mg/Kg of the final composition, copper in an amount from about 1 to 7.3 mg/Kg of the final composition, manganese in an amount from 1 to 5.0 mg/Kg of the final composition, zinc in an amount from 10 to 80 mg/Kg of final product, iodine in an amount from about 0.1 to 0.88 mg/kg of final composition, and selenium in an amount from 0.05 to 0.35 mg/Kg of final composition.

11. The hard chew matrix of claim 2 wherein the polyunsaturated fatty acids are selected from the group consisting of krill oil, krill meal, krill extracts, fish oil, fish meal, plant oils, omega-3 fatty acids and omega-6 fatty acids.

12. The hard chew matrix of claim 2 wherein the polyunsaturated fatty acids are present in an amount from about 0.001% to about 25% w/w of the final composition.

13. The hard chew matrix of claim 2 wherein the polyunsaturated fatty acids are present in an amount from about 1.0% to about 20.0% w/w of the final composition.

14. The hard chew matrix of claim 2 wherein the antioxidant is selected from the group consisting of astaxanthin, alpha-tochopherol, alpha-tochopherol acetate, butylated hydroxytoluene, ascorbic acid, tocopherol and propyl gallate.

15. The hard chew matrix of claim 2 wherein the antioxidant is present in an amount from about 0% to about 0.3% w/w of the final composition.

16. The hard chew matrix of claim 1 wherein the inner core matrix further comprises an anti-inflammatory agent selected from the group consisting of turmeric, garlic, cinnamon, ginger, Roman chamomile, Echinacea, red clover, goldenseal, Vitex (Chaste tree), black pepper, and clove.

17. The hard chew matrix of claim 16 wherein the anti-inflammatory agent is selected from turmeric and extracts of turmeric containing curcumin.

18. The hard chew matrix of claim 2 wherein the vitamins are present in an amount between about 0% and 10% w/w of the final composition.

19. The hard chew matrix of claim 2 wherein the vitamins selected from water soluble and fat-soluble vitamins.

20. The hard chew matrix of claim 19 wherein the water-soluble vitamins are selected from the group consisting of any of the vitamin B group and vitamin C in amounts from 0 to 10% w/w of the composition.

21. The hard chew matrix of claim 19 wherein the fat-soluble vitamins are selected from the group consisting of vitamin A, vitamin E, vitamin D and vitamin K in amounts from 0 to 10% w/w of the composition.

22. The hard chew matrix of claim 1 wherein the inner core matrix further comprises a plasticizer, wherein the plasticizer is glycerol present in an amount between about 2% and 25% of the final composition.

23. The hard chew matrix of claim 1 wherein the inner core matrix further comprises a nutraceutical present in an amount ranging from about 0.001% w/w to 50% w/w of the composition.

24. The hard chew matrix of claim 23 wherein the nutraceutical is selected from the group consisting of flavonoids, isoprenoids, proteins, bioflavonoids, carotenoids, glucosamine and chondroitin sulfate.

25. The hard chew matrix of claim 1 wherein the inner core matrix further comprises a flavoring agent.

26. The hard chew matrix of claim 1 wherein the outer core matrix is selected from the group consisting of flour, starch, glycerol, carboxymethyl cellulose, flour, xanthan gum, brewer's yeast, and flaxseed.

27. The hard chew matrix of claim 26 wherein the flour is selected from the group consisting of potato, wheat, corn, oat, soy, barley and rice flour.

28. The hard chew matrix of claim 27 wherein the starch is selected from potato, corn, wheat, oat, soy, barley or rice.

29. The hard chew matrix of claim 1 wherein the inner core matrix further comprises a preservative selected from the group consisting of potassium sorbate, methylparaben, propylparaben, sodium benzoate, calcium propionate and sorbic acid.

30. The hard chew matrix of claim 29 wherein the preservative is present in the inner core matrix in an amount between 0% and 1% by weight of the composition.
